(19) United States
(12) Patent Application Publication
(10) Pub. No.: US 2020/0096448 A1
(43) Pub. Date: Mar. 26, 2020

(54) METHOD AND DEVICE FOR PROVIDING A CELL LINE HAVING A DESIRED TARGET PROTEIN EXPRESSION

(71) Applicant: FRAUNHOFER-GESELLSCHAFT ZUR FÖERDERUNG DER ANGEWANDTEN FORSCHUNG E.V., Müenchen (DE)

(72) Inventors: Arnold GILLNER, Roetgen (DE); Nadine NOTTRODT, Aachen (DE); Martin WEHNER, Herzogenrath (DE); Dominik RIESTER, München (DE); Anke BURGER-KENTISCHER, Stuttgart (DE); Monika BACH, Leinfelden-Echterdingen (DE); Eva BRAUCHLE, Tübingen (DE); Katja SCHENKE-LAYLAND, Tübingen (DE); Benjamin GREINER, Königswinter (DE); Andreas PIPPOW, Köln (DE); Phuong-Ha NGUYEN, Bonn (DE); Katharina HOFER-SCHMITZ, Graz (AT)

(21) Appl. No.: 16/609,146

(22) PCT Filed: Apr. 6, 2018
(86) PCT No.: PCT/EP2018/058883

§ 371 Date: Oct. 28, 2019

(30) Foreign Application Priority Data
Apr. 28, 2017 (DE) 10 2017 207 262.8

(51) Int. Cl. G01N 21/65; G01N 33/68; G16B 40/30

(52) U.S. Cl. CPC G01N 21/658; G16B 40/30; G01N 33/6803

(57) ABSTRACT

The invention relates to a method for providing a cell line having a desired target protein expression and to a device for selecting cell lines having a desired target protein expression.

The invention relates to a method for providing a cell line having a desired target protein expression and to a device for selecting cell lines having a desired target protein expression.

The production of efficient production cell lines (high-producer cells) is an essential step in the biotechnological production of active pharmaceutical ingredients. Nowadays, the selection of cells which have a particularly high expression rate of a desired target protein, takes place via complex, in particular time-consuming and cost-intensive, mostly screening methods based on immunological methods for determining the expressed amount of the target protein. There is therefore a great need for rapid automation methods for the selection of specific cells depending on the expression of the desired target proteins in order to establish more cost- and time-saving production methods of suitable production cell lines.

In the prior art, cells transfected for the selection of suitable production cell lines are analyzed by fluorescence methods for the expression performance of the target protein. For this fluorescence markers are conventionally used which mark the target protein in the supernatant of the cell cultures and can be detected by standard fluorescence analysis methods. On the one hand, however, this requires knowledge of the structure of the target protein and, on the other hand, the presence of a suitable marker system. In common methods, the clones are for example marked with fluorescence-marked antibodies which bind to the target protein, and the segregated amount of target protein is determined by means of immunofluorescence methods. After analysis of the cells selected by means of fluorescence methods, these cells are isolated by means of manual techniques or by means of so-called cell pickers from the cell colony and are isolated as a clone for further expansion. For the automatic selection of high-producer cells, commercial devices are available with which small cell colonies can be removed from culture dishes and transferred. For this, metal needles are used, for example, which are pressed onto the cell colony, so that the adhering cells can be removed in a targeted manner. Alternatively, microdispensers are offered, which can take up the cells with liquid via a glass capillary and can settle them elsewhere. These systems are also commonly equipped with facilities for fluorescence analysis and image processing to identify the single cell clones. The positioning of the needles and cannulas for the removal of single clones can thereby be controlled automatically via the image processing software. In addition, bright field microscopy or phase contrasting methods can be used for the analysis of the cell morphology. However, with the systems known up to now in the prior art, it is not possible to extract cells already analyzed for their protein expression in a targeted manner from a single culture.

A disadvantage of the previous methods is therefore in particular the fact that the target cells must be marked for determining the expression rate for the target protein and the selection techniques described in the prior art up to now do not have the necessary analysis technique to isolate single cells analyzed on their protein expression. In addition, until now, the cell selection can only take place after sufficient expression of the target protein in the supernatant of the cell. In conventional methods, the cultivation of the clones takes several weeks up to months.

The present invention is therefore based on the technical problem of providing a method for providing a cell line having a desired target protein expression, in particular such a method which overcomes the aforementioned disadvantages. The invention is also based on the technical problem of providing a device for selecting cell lines having a desired target protein expression.

The present invention solves the underlying problem by the teaching of the independent claims.

The method according to the invention for providing a cell line having a desired target protein expression thereby comprises the following steps:
a) providing cells each having at least one nucleotide sequence encoding at least one target protein,
b) characterizing the protein expression of the cells provided in step a) at the single cell level by means of Raman spectroscopy,
c) selecting at least one cell having a desired target protein expression,
d) transferring the at least one cell selected in step c) into an expansion medium,
e) cultivating the at least one selected cell in an expansion medium,
f) characterizing the quantitative protein expression of the at least one cell cultivated in step e) on a single cell plane by means of time-resolved Raman spectroscopy, and
g) selecting a cell line having the desired target protein expression.

In a particularly preferred embodiment, the method steps a) to g) are carried out in the order indicated. In a particularly preferred embodiment, the present method consists of the present method steps, that is, no further method steps take place between the individual method steps, preferably, neither before nor after performing the method steps a) to g) without intermediate steps, further method steps for providing the cell line having the desired target cell expression cell line are provided.

Accordingly, the method according to the invention advantageously provides a combination, in particular of marker-free characterization of the protein expression of cells at the single cell level according to method step b) and one, in particular laser-based, individual cell selection according to method step c), according to method step b), the expression of a target protein already within one single cell can be detected and wherein the cell thus analyzed can be selected and separated directly in a single step for the further production of a desired target protein expression cell line.

The invention makes it possible in an efficient and simple manner to provide a cell line having a desired target protein expression with high precision and reliability, in particular in a short time.

By “desired target protein expression” is meant, in the context of the present invention, that an expression of a target protein targeted for a particular use of the cell line, for example, a certain targeted, e.g. high or low expression amount or a particularly targeted, e.g. high or low, expression rate of the target protein, is realized from a provided cell line, in particular stable and over a longer period.

In the context of the present invention, the term “target protein” refers to the protein whose expression in a cell is of interest. According to the invention, it can be both a protein occurring naturally in the provided cells and a protein whose presence and/or expression was induced artificially in a targeted manner in the provided cells.

In the context of the present invention, a “nucleotide sequence” is understood to mean the sequence of the nucleotides of a nucleic acid. The nucleotide sequence is preferably a DNA sequence or an RNA sequence, in particular a DNA sequence.

According to the invention, the term “cells” is understood to mean that at least two cells, preferably several, in particular at least 10³, 10⁴, 10⁵, 10⁶, 10⁷, 10⁸ or more cells are present. The cells provided in step a) can represent a proportion of a cell population having an even greater number of cells. Thus, it is conceivable that in step a) a cell population is present, of which only a certain number of cells each have at least one nucleotide sequence which encodes at least one target protein and the remaining cells do not have such a nucleotide sequence.

In the context of the present invention, “unsupervised” means that Raman spectra obtained in step b) are unlabeled with respect to the protein expression of the cells provided in step a), that is, the expected spectra of the expression patterns are unknown. In the context of the present invention, “partially monitored” means that Raman spectra obtained in step b) are partially labeled with respect to the protein expression of the cells provided in step a), that is, the expected spectra of the expression patterns are partially known.

The invention provides, in method step a), to provide cells, wherein the provided cells each have at least one nucleotide sequence which encodes at least one target protein.

In a preferred embodiment, the cells provided in step a) are animal cells, preferably mammalian cells, preferably rodent cells, preferably mouse cells, preferably hamster cells, preferably cells of the Chinese dwarf hamster (Cricetulus griseus), in particular ovary cells (CHO cells) of the Chinese dwarf hamster, preferably NSO cells, preferably PERC6 cells, preferably HeLa cells, preferably HEK293 cells, preferably kidney cells (BHK cells) of the Chinese dwarf hamster.

Preferably, the cells provided in step a) are mammalian cells. Preferably, the cells provided in step a) are not mammalian cells.

Preferably, the cells provided in step a) are insect cells. Preferably, the cells provided in step a) are not insect cells.

Preferably, the cells provided in step a) are microorganisms. Preferably, the cells provided in step a) are not microorganisms.

Preferably, the cells provided in step a) are bacterial cells. Preferably, the cells provided in step a) are not bacterial cells.

In a preferred embodiment, the at least one nucleotide sequence encoding at least one target protein is arranged on a plasmid.

In a preferred embodiment, the at least one nucleotide sequence encoding the at least one target protein is stable or transiently integrated into the genome of the cells. The at least one nucleotide sequence encoding at least one target protein is preferably stably integrated into the genome of the cells. Preferably, the at least one nucleotide sequence encoding at least one target protein is transiently integrated into the genome of the cells.

The at least one nucleotide sequence encoding at least one target protein preferably encodes exactly one target protein. The at least one nucleotide sequence encoding at least one target protein preferably encodes exactly two, preferably exactly three, preferably exactly four, preferably exactly five target proteins. Preferably, the at least one nucleotide sequence encoding at least one target protein encodes at least two target proteins, preferably at least three, at least four or at least five target proteins. Preferably, the at least one nucleotide sequence encoding at least one target protein encodes any number of target proteins.

In a preferred embodiment, prior to step a), a transfection of cells with at least one nucleotide sequence takes place, which encodes for at least one target protein.

Preferably, the methods known to those skilled in the art are used for the transfection of the cells. Preferably, the method for the transfection of cells selected from calcium phosphate precipitation, lipofection, cationic polymer transfection, microinjection, electroporation, particle gun, magnetofection, sonoporation, transfer infection, and antibody-mediated transfection.

In a preferred embodiment, a selection of transfected cells takes place before step a). Preferably, no selection of transfected cells takes place before step a).

Preferably, all cells provided in step a) each have at least one nucleotide sequence which encodes at least one target protein. Preferably, only a portion of the cells provided in step a) each have at least one nucleotide sequence which encodes at least one target protein.

According to the invention, in method step b), a characterization of the protein expression of the cells provided in step a) at the single cell level is carried out by means of Raman spectroscopy. Characterizing the protein expression of cells at the single cell level in the context of the present invention means that an analysis of the expression, for example the expression rate or the expression amount of a particular protein is performed individually for a particular single cell.

The optical system used for the Raman spectroscopy provided according to the invention comprises a Raman spectrometer and at least one microscope in a preferred embodiment. In the context of the present invention, a Raman spectrometer comprises at least one light source, in particular at least one excitation light source, in particular at least one laser, and at least one detector. In addition, microfluidic systems can be realized with direct light coupling via optical fibers brought directly to the cells, which permit a high numerical aperture or collection efficiency of the signal detection without adding further optical elements.

The Raman spectroscopy used to analyze protein expression advantageously makes it possible to determine high resolution molecular structures at the single cell level. The examination of cells on a single cell level is thereby realized in particular by the coupling of the Raman spectrometer to a microscope or light collector with high numerical aperture. In Raman spectroscopy, molecular vibrations are detected, making it possible to identify solids, certain liquids and/or gases, and biomolecules without the need for a marker. Specific vibration patterns of the proteins contained in the cells or in the supernatant can be excited with a laser in the visible or near-infrared region, without negatively affecting cell viability. The backscattered light has a slightly changed wavelength due to energy loss. In this way, differences and specific features in the protein composition of a single cell can be determined with the help of the detected spectra. The analysis of the cells thereby preferably takes place within an optical system in which the cells are irradiated in a microscope beam path and the backscattered and frequency-shifted light is analyzed.

In a particularly preferred embodiment of the present invention, Raman spectroscopy is Surface Enhanced Raman Spectroscopy (SERS).

In a preferred embodiment, the characterization of the protein expression of the cells provided in step a) at the single cell level in step b) accordingly takes place by means of surface-enhanced Raman spectroscopy (SERS), preferably by means of quantitative surface-enhanced Raman spectroscopy.

Surface-enhanced Raman spectroscopy (SERS) provides much more intense signals compared with the classical Raman effect, and is particularly useful when the intensity of the signals detected in Raman spectroscopy is very low, for example due to the small amount of material. SERS is based on the local field enhancement of electrically conductive particles or nanostructures and makes it possible to increase the intensity of the obtained Raman signal by a factor of 10³ to 10⁶ compared with classic Raman spectroscopy. Thus, for example, colloidal gold layers can be used as carrier substrates, which can be used in particular for secreted proteins, such as antibodies. Another possibility is to introduce copper, platinum, palladium, silver, or gold nanoparticles into cells to enhance intracellular signals.

The present invention thus advantageously makes it possible in step b) to characterize the protein expression of single cells by means of Raman spectroscopy, preferably by means of surface-enhanced Raman spectroscopy (SERS).

In a preferred embodiment, no markers, in particular no fluorescence markers, are used in the method according to the invention. The method according to the invention is preferably marker-free.

In a further preferred embodiment of the present invention, after step b), a classification of the cells characterized in step b) is carried out by means of principal component analysis (PCA).

In a further preferred embodiment of the present invention, after step b), a classification of the cells characterized in step b) takes place by means of unsupervised or partially monitored ensemble machine learning methods and/or analysis by means of neural networks.

Preferably, in step b), the Raman spectra separating different spectral signatures (which correlate with different target protein expressions) are separated by Ensemble Machine Learning methods. For this, the noisy spectra are preprocessed with suitable methods (for example with baseline correction, wavelets or principal component analysis (PCA)). In particular, unsupervised and partially monitored ensemble machine learning methods are used to separate the spectra. The advantage of this method is that, in the examined set of spectral signatures, such signatures are found which are not known a priori and correspond to the desired target protein expression.

According to the invention, in step c) at least one cell is selected which has a desired target protein expression according to the characterization carried out in step b). Preferably, the selection of the at least one cell having the desired target protein expression is automated.

According to the invention, the at least one cell selected in step c) is transferred into an expansion medium in step d).

The present invention provides in a preferred embodiment, and advantageously in step d), to transfer single cells selected in step c) into an expansion medium respectively associated with the single cells.

In a preferred embodiment, the transfer of the at least one selected cell into an expansion medium according to step d) takes place by means of a laser-based cell transfer technique.

In a particularly preferred embodiment, the laser-based cell transfer technique is a laser-induced forward transfer (lift) method which is also called laser-assisted bioprinting for biological materials.

In the Laser Induced Forward Transfer (LIFT) method, a small amount of material is transferred from a transfer carrier to a target substrate by means of a targeted laser pulse. The transfer carrier usually consists of three layers, a transparent carrier layer for the laser light, an absorber and a transfer material layer. In the prior art, however, variants are also known in which absorbent carrier layers are used instead of absorber layers. The absorber layer/absorbing carrier layer is briefly heated by a laser pulse and an extensional wave or a vapor bubble is generated, which triggers the transport of a small amount of material similar to a jet print head. The resulting drop or particle can be placed on a receiver carrier, e.g. a slide or a microtiter plate, at a distance of about 0.1 to 1 mm.

The materials to be transferred can thereby be present as liquid, highly viscous or solid substances. The materials to be transferred can also be present as a combination of different substances, as for example liquids and cells. With an optical target recognition technology, single cells can be specifically targeted and transmitted without decisively influencing the cell vitality. An advantage of the LIFT method is the integratability of further methods, for example light microscopy and Raman spectroscopy, with which cells can be continued to be examined automatically.

In a preferred embodiment, the laser pulse energy in the LIFT method is 1 μJ to 20 μJ, preferably 1 μJ to 15 μJ, preferably 1 μJ to 10 μJ, preferably 2 μJ to 10 μJ, preferably 3 μJ to 10 μJ, preferably 5 μJ to 10 μJ, preferably 6 μJ to 10 μJ, preferably 8 μJ to 10 μJ.

In a further preferred embodiment, the diameter of the laser focal spot in the LIFT method is 10 μm to 200 μm, preferably 15 μm to 150 μm, preferably 20 μm to 100 μm, preferably 25 μm to 90 μm, preferably 30 μm to 80 μm, preferably 40 μm to 80 μm, preferably 50 μm to 70 μm.

The carrier layer of the transfer carrier used in the LIFT method preferably consists of glass, preferably of quartz, or of plastic.

Preferably, the absorber layer of the transfer carrier used in the LIFT method consists of titanium, gold or another substance absorbing the laser wave length. The thickness of the absorber layer of the transfer carrier used in the LIFT method is preferably 5 nm to 300 nm, preferably 5 nm to 250 nm, preferably 5 nm to 200 nm, preferably 5 nm to 150 nm, preferably 5 nm to 120 nm, preferably 5 nm to 100 nm, preferably 5 nm to 80 nm, preferably 5 nm to 60 nm, preferably 10 nm to 50 nm, preferably 10 nm to 40 nm, preferably 10 nm to 30 nm, preferably 10 nm to 20 nm.

In a preferred embodiment of the present invention, the transfer carrier does not have an absorber layer. Preferably, the transfer material layer serves as an absorber layer. When using the transfer material layer as the absorber layer, IR laser sources with a wavelength of >3 μm are preferably used.

Preferably, the distance between the transfer carrier and the receiver carrier in the LIFT method is 20 μm to 2000 μm, preferably 25 μm to 1800 μm, preferably 30 μm to 1600 μm, preferably 40 μm to 1400 μm, preferably 50 μm to 1200 μm, preferably 60 μm to 1100 μm, preferably 70 μm to 1100 μm, preferably 80 μm to 1000 μm, preferably 90 μm to 1000 μm, preferably 100 μm to 1000 μm, preferably 200 μm to 900 μm, preferably 300 μm to 800 μm, preferably 400 μm to 800 μm.

According to the invention, in step e), the cultivating of the at least one cell selected in step c) takes place in an expansion medium. The at least one cell selected in step c) is cultivated in an expansion medium in step e) for preferably at least one hour, preferably at least 2 hours, preferably at least 4 hours, preferably at least 6 hours, preferably at least 8 hours, preferably at least 10 hours, preferably at least 12 hours, preferably at least 24 hours, preferably at least 2 days, preferably at least 3 days, preferably at least 4 days, preferably at least 5 days, preferably at least 6 days, preferably at least 7 days.

In a preferred embodiment of the present invention, the cultivating of the at least one cell selected in step c) in a protein and serum-free expansion medium takes place in step e). In a preferred embodiment of the present invention, the cultivation of the at least one cell selected in step c) in a protein and serum-containing expansion medium takes place in step e).

According to the invention, in step f), the quantitative protein expression of the at least one cell cultivated in step e) is characterized on a single cell level by means of SERS and/or time-resolved Raman spectroscopy.

In a preferred embodiment of the present invention, the characterization of the quantitative protein expression of the at least one cell cultivated in step e) takes place on the single cell level in step f) in the expansion medium.

According to the invention, the selection of one, preferably at least one, cell line having the desired target protein expression takes place in step g). Preferably, exactly one cell line having the desired target protein expression is selected in step g). Preferably two, preferably three, preferably four, preferably five, preferably six, cell lines which have the desired target protein expression are selected in step g). It is particularly preferred in step g) to select any number of cell lines having the desired target protein expression.

The present invention also relates to a device for the selection of cell lines, comprising

i) at least one optical system for Raman spectroscopy at the single cell level,
ii) at least one system for the transfer of selected cells.

In a preferred embodiment, the at least one optical system according to i) is a system for surface-enhanced Raman spectroscopy on a single cell level.

In a further preferred embodiment, the at least one system for the transfer of selected cells is a Laser Induced Forward Transfer (LIFT) system.

Preferably, the statements made in context with the method according to the invention or the statements and listed embodiments preferred according to the invention apply mutatis mutandis also to the device for the selection of cell lines having a desired target protein expression.

Further advantageous embodiments of the invention will become apparent from the dependent claims.

The present invention will be explained in more detail below with reference to the figures.

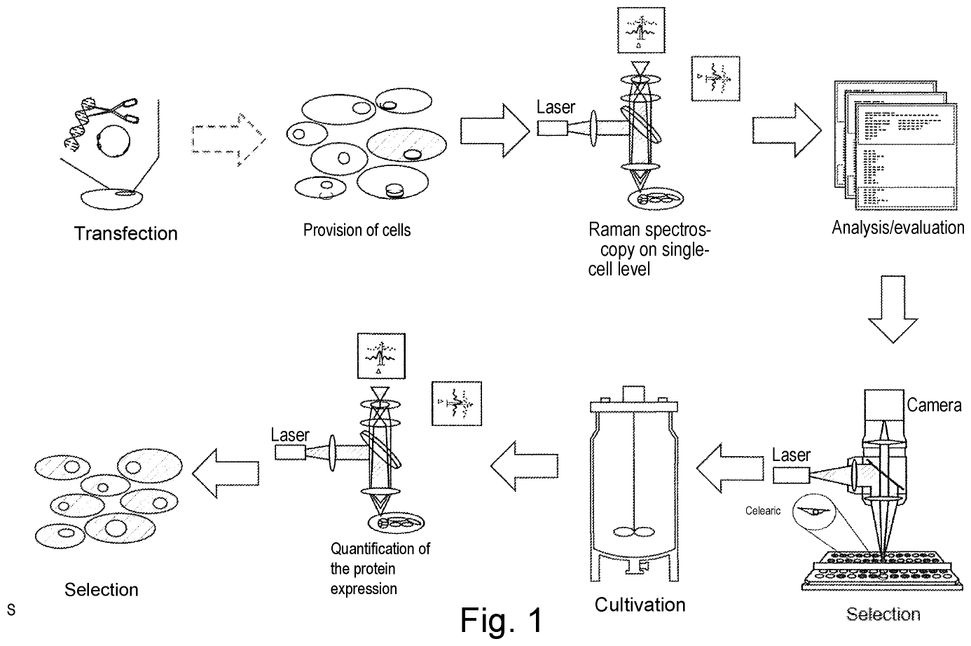

FIG. 1 schematically shows the method steps of the method according to the invention for providing a cell line having a desired target protein expression. Thereby, in a first step, cells are provided (provision of cells), which have at least one nucleotide sequence which encodes at least one target protein. In a preferred embodiment of the present invention, the provided cells can be obtained in an upstream method step by transfection (transfection) of cells with at least one nucleotide sequence which encodes at least one target protein. The protein expression of the provided cells is then characterized on the single cell level by means of Raman spectroscopy (Raman spectroscopy at the single cell level). The resulting Raman spectra are then analyzed (analysis/evaluation), at least one cell having a desired target protein expression is selected (selection) and transferred to an expansion medium. The at least one selected cell is then cultivated in an expansion medium (cultivation). In a further step, a characterization of the quantitative protein expression (quantification of the protein expression) of the cultivated cells on the single cell level takes place by means of time-resolved Raman spectroscopy and the selection (selection) of at least one cell line which has the desired target protein expression.

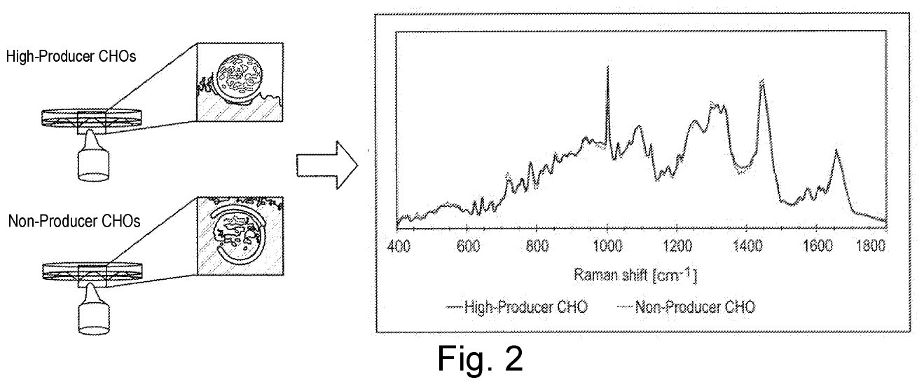

FIG. 2 shows different spectra of CHO cells with different target protein expression by means of single-cell Raman spectroscopy, namely high-producer CHO cells (high expression of the target protein) on the one hand and non-producer CHO cells (no expression of the target protein) on the other hand.

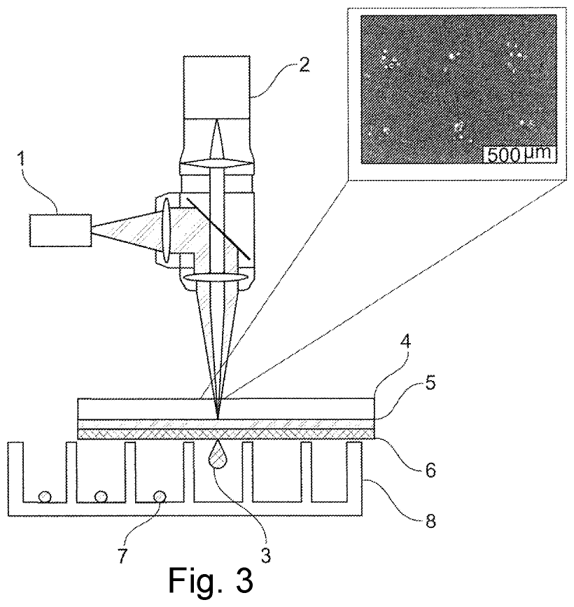

FIG. 3 shows the LIFT method preferably used for the transfer of selected single cells. Thereby, by means of a laser (1), a laser pulse is effected, which passes through the carrier layer (4) of a transfer carrier (4, 5, 6) and leads to a short-term local heating of the absorber layer (5), whereby an extensional wave or vapor bubble is generated in the transfer layer (6), which makes it possible to transfer cells (3) in a targeted manner to a receiver carrier (8) over a short distance and to separate cells selected in this way (7). By means of an optical target recognition technique (2), single cells (3) can be intentionally targeted and transmitted individually with the method.

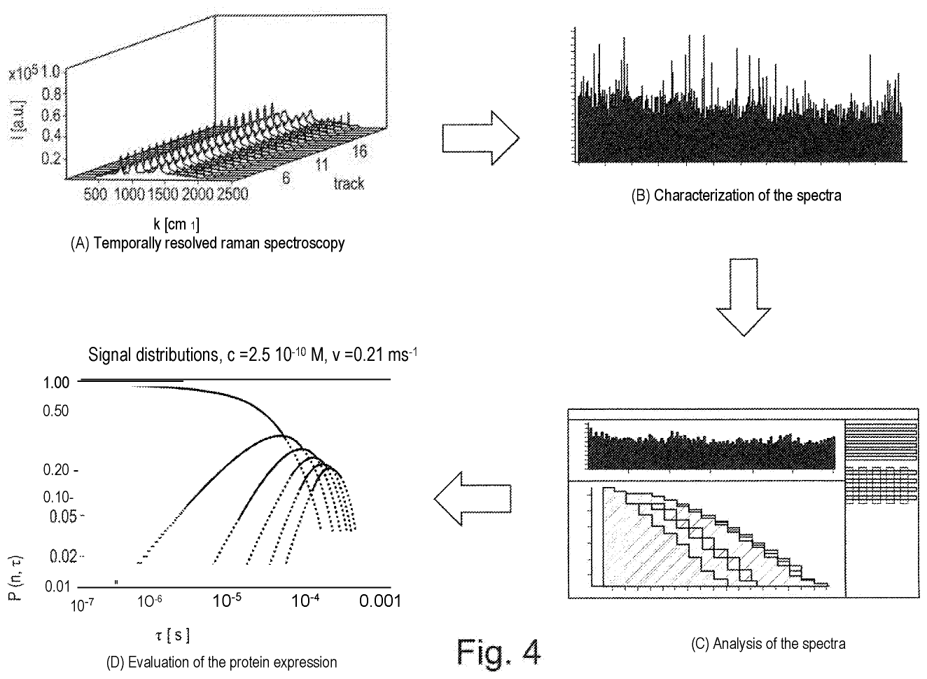

FIG. 4 schematically shows a method for characterizing the quantitative protein expression at the single cell level. For this, a large number of Raman spectra of a single sample with low exposure times is first recorded in the form of a time series ((A), time-resolved Raman spectroscopy). Relevant features are then determined in the obtained spectra and a time series is generated for the respective pixel position ((B), characterization of the spectra). In a further step, the time series are histogrammed with variation of the bin widths ((C), analysis of the spectra) and the data thus obtained are finally plotted as a function of time, in order to obtain sample-specific information on the target protein expression ((D), evaluation of the protein expression).

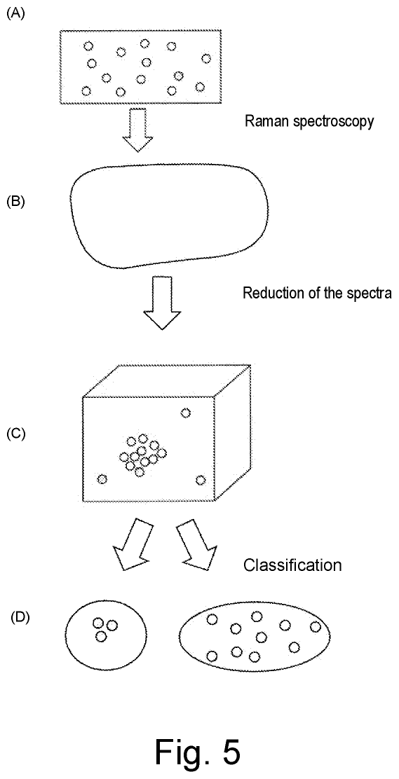

FIG. 5 shows a method for classifying the cells characterized by Raman spectroscopy in the method according to the invention. Thereby, single cells located on a carrier are first characterized by means of Raman spectroscopy ((A), Raman spectroscopy). Then, the feature space dimensions of the obtained Raman spectra are reduced ((B), reduction of the spectra) and classified by means of ensemble machine learning methods ((C), classification), so that finally, originating from the recorded Raman spectra, a classification of the characterized cells is possible depending on the target protein expression (D).

1. A method of providing a cell line having a desired target protein expression, the method comprising the steps of:
a) providing cells each having at least one nucleotide sequence encoding at least one target protein,
b) characterizing the protein expression of the cells provided in step a) at the single cell level by means of Raman spectroscopy,
c) selecting at least one cell having a desired target protein expression,
d) transferring the at least one cell selected in step c) into an expansion medium,
e) cultivating the at least one selected cell in an expansion medium,
f) characterizing the quantitative protein expression of the at least one cell cultivated in step e) on a single cell plane by means of time-resolved Raman spectroscopy, and
g) selecting a cell line having the desired target protein expression.

2. The method according to claim 1, wherein the method is marker-free.

3. The method according to claim 1, wherein prior to step a) a transfection of cells with at least one nucleotide sequence takes place, which encodes at least one target protein.

4. The method according to claim 1, wherein the characterization of the protein expression of the cells in step b) takes place by means of surface-enhanced Raman spectroscopy.

5. The method according to claim 1, wherein after step b), a classification of the cells characterized in step b) takes place by means of principal component analysis (PCA).

6. The method according to claim 1, wherein after step b) a classification of the cells characterized in step b) takes place by means of ensemble machine learning methods and analysis by means of neural networks.

7. The method according to claim 6, wherein the ensemble machine learning methods are unmonitored or partially supervised.

8. The method according to claim 1, wherein the transferring in step d) takes place by means of laser induced forward transfer (LIFT).

9. The method according to claim 1, wherein the characterization of the quantitative protein expression in step f) takes place in the expansion medium.

10. A device for selecting cell lines having a desired target protein expression, comprising:
at least one optical system for Raman spectroscopy at the single cell level, and
at least one system for the transfer of selected cells.

11. The device according to claim 10, wherein the at least one optical system is a system for surface-enhanced Raman spectroscopy at the single cell level.

12. The device according to claim 10, wherein the at least one system for the transfer of selected cells is a Laser Induced Forward Transfer (LIFT) system.
